(19) United States
(12) Patent Application Publication
(10) Pub. No.: US 2026/0301912 A1
(43) Pub. Date: Oct. 1, 2026

(54) SYSTEMS AND METHODS FOR DOSING EXTERNALLY POWERED CORTICAL STIMULATION (XCS) THERAPY

(71) Applicant: MOTIF NEUROTECH, INC., Houston, TX (US)

(72) Inventors: Brandon Thio, Houston, TX (US); Benjamin Ferleger, Houston, TX (US); Ameya Nanivadekar, Houston, TX (US); Landan Mintch, Houston, TX (US); Amanda Singer, Houston, TX (US); Elizabeth Commissaris, Houston, TX (US); Jacob Robinson, Houston, TX (US); Steven Goetz, Houston, TX (US)

(73) Assignee: MOTIF NEUROTECH, INC., Houston, TX (US)

(21) Appl. No.: 19/559,861

(22) Filed: Mar. 6, 2026

Related U.S. Application Data
(60) Provisional application No. 63/777,564, filed on Mar. 25, 2025.

(51) Int. Cl. G16H 20/30; A61B 5/00; A61N 2/02

(52) U.S. Cl. CPC G16H 20/30; A61B 5/4836; A61N 2/02

(57) ABSTRACT

Externally powered stimulation therapies may be applied to a targeted region to modulate a physiological activity within a subject and to address one or more physiological conditions. The present disclosure relates to configuring a set of selection parameters based on data from a non-invasive technique, and rhythmic parameters or event-based parameters derived from physiological data of the subject for modulation of the physiological activity. The techniques, as disclosed herein, may initiate an externally powered cortical stimulation (XCS) therapy and a regenerated XCS therapy to the targeted region of the subject through a stimulation electrode, based on the set of selection parameters. The regenerated XCS therapy may be initiated after reconfiguring the set of selection parameters based on feedback from the subject following the delivery of the XCS therapy.

### CROSS-REFERENCE TO RELATED APPLICATIONS

This application claims the benefit of U.S. Provisional Patent Application No. 63/777,564 filed on Mar. 25, 2025. The entire disclosures of the aforementioned applications are incorporated by reference herein in their entireties for all purposes.

### BACKGROUND

Neuromodulation is an evolving field of medical science that involves an application of targeted electrical, chemical or magnetic stimuli to a nervous system to regulate neural or nerve activities. One goal is to modulate neural activity by stimulating specific regions of the brain, spinal cord, or peripheral nerves. Empirical research suggests that such a therapeutic approach appears to show promising outcomes while treating a range of conditions, including chronic pain, neurological disorders, and psychiatric diseases, particularly when conventional therapies such as medications or surgery may not be as effective as desired or send up producing significant undesirable side effects. Neuromodulation therapies are generally categorized into invasive and non-invasive therapeutic methods. While non-invasive methods may utilize external devices, invasive methods may need surgical implantation of devices that directly deliver stimulation to a nervous system.

Externally powered cortical stimulation (XCS) is one such invasive neuromodulation technique that may electrically stimulate a brain using epidural cortical stimulation. In XCS, millimeter-sized neuromodulation devices are implanted into skull via a burr hole procedure. A magnetic coil is employed to power and digitally program a device that allows for precisely delivering stimulation to targeted areas of a brain. XCS has shown a significant potential in treating various neurological and mental health disorders by providing targeted neuromodulation. However, like all other neuromodulation therapies, various effects of XCS are dose-dependent, meaning that different parameters controlling stimulation, such as intensity, frequency and duration, may be carefully selected to achieve positive therapeutic outcomes.

Success of XCS, like other invasive neuromodulation therapies, may depend on setting of stimulation parameters. Since a response to XCS can fluctuate over time and in response to changes in a subject's physiological condition or disease progression, continuous monitoring and real-time adjustments may be needed for maintaining therapeutic efficacy. Inadequate or excessive stimulation can lead to suboptimal results or even adverse outcomes. Therefore, fine-tuning various parameters of stimulation to match specific needs of each individual subject may enhance effectiveness of a therapy. This need for individualized dosing makes it relevant to frequently assess an impact of XCS in real-time and adjust stimulation parameters accordingly.

### SUMMARY

Some aspects of the present disclosure relate to techniques for initiating a XCS therapy, via an invasive technique, based on data from a non-invasive technique applied to a subject. The data from the non-invasive technique may be used to calibrate a set of selection parameters associated with the invasive technique to initiate the XCS therapy (for example) an externally powered cortical stimulation (XCS) be delivered to a cortical region of the subject. The data from a non-invasive technique may include, but is not limited to, motor threshold, recruitment curves and normalized peaks of the recruitment curves based on a stimulus or stimulation amplitudes applied to a subject using the non-invasive technique. The stimulus associated with the non-invasive technique may be delivered to the subject over a motor cortex region and a motor response of the subject may be identified in one or more regions controlled by the motor cortex including (for example) thumb, hand or any other body part. An intensity of the stimulus that evokes a motor response in the identified region controlled by the motor cortex with a 50% probability may be defined as the motor threshold.

After determining the motor threshold, the non-invasive technique may be applied over a cortical region of the subject, other than the motor cortex, to map the recruitment curves by sweeping the simulation amplitudes between 0% and 100% of the intensity associated with the non-invasive technique. The responses evoked by sweeping the stimulation amplitudes may be recorded using one or more electroencephalography (EEG) electrodes. Based on the responses evoked, normalized peaks of the recruitment curves at the motor threshold may be identified.

Based on the data from the non-invasive technique, an invasive technique may be employed that may comprise implanting a stimulation electrode over the cortical region of the subject to deliver the XCS. The placement of the stimulation electrode may be configured using an XCS implantation module through a surgical procedure for implantation within a subject. The XCS may be delivered, via the stimulation electrode, to gather data associated with the invasive technique including, but is not limited to, recruitment curves and normalized peaks of the recruitment curves based on stimulation amplitudes applied to the subject. The recruitment curves may be mapped by sweeping the simulation amplitudes between 0 and 15 mA of the intensity associated with the invasive technique and evoked responses may be recorded using the one or more EEG electrodes. Based on the responses evoked, normalized peaks of the recruitment curves associated with the invasive technique may be identified at the motor threshold.

The normalized peaks of the recruitment curves associated with the non-invasive technique and the invasive technique may be matched to identify the stimulation amplitudes at which the evoked response peaks from the non-invasive technique align most closely with those generated by the invasive technique. Based on matching of the normalized peaks, an initial stimulation amplitude for the invasive technique may be determined—the amplitude that may generate normalized evoked response peaks closely matching those of the non-invasive evoked response peaks. The initial stimulation amplitude for the invasive technique may form a part of the set of selection parameters to initiate the XCS.

The cortical region, other than the motor cortex, may include (for example) sensory cortex, visual cortex, auditory cortex, prefrontal cortex (e.g., left and right dorsolateral prefrontal cortex (DLPFC)) or language areas (e.g., Broca's and Wernicke's areas). In some aspects, stimulation amplitudes associated with the non-invasive technique and the invasive technique may be applied over the same cortical region of a subject. In some other aspects, stimulation amplitudes associated with the non-invasive technique and the invasive technique may be applied over different cortical region of a subject. The cortical region may be selected based on therapeutic requirements of a subject and the intended outcomes of a XCS therapy.

In some aspects, the non-invasive technique comprises transcranial magnetic stimulation (TMS) delivered using TMS coils. In some other aspects, the non-invasive technique may include, but is not limited to, transcranial direct current stimulation (tDCS), transcranial ultrasound stimulation (TUS), transcranial focused ultrasound (FUS), functional near-infrared spectroscopy (fNIRS) or magnetic seizure therapy (MST).

Some aspects of the present disclosure relate to additional techniques for initiating an XCS, via an invasive technique, based on physiological data of a subject that may be obtained from one or more wearable assemblies. The one or more wearable assemblies configured to obtain the physiological data of the subject may include, but are not limited to, a head-mounted device, one or more wrist-worn devices, Holter monitors, electromyography (EMG) patches, pulse oximeters, blood pressure monitors, glucose monitors and smart monitors. Each wearable assembly may comprise of a data acquisition assembly with one or more clusters of sensors and/or sensor electrodes designed to capture the physiological data of a subject. The disclosed techniques may further include externally powering and digitally programming the stimulation electrode associated with the invasive technique to initiate the XCS via a magnetic coil associated with a wearable assembly of the one or more wearable assemblies including (for example) a head-mounted device. The magnetic coil may be, may include or may be replaced or supplemented with a magnetic transceiver. In some aspects, the head-mounted device may comprise one or more EEG electrodes to record the responses evoked by a non-invasive technique and/or an invasive technique. The head-mounted device may wirelessly receive from other devices of the one or more wearable devices the physiological data of a subject.

The physiological data collected by the one or more wearable assemblies may include, but is not limited to, one or more of: neural data, cardiovascular data, respiratory data, movement and activity data, environmental data, demographic data (e.g., age, gender, race), anthropometric data (e.g., weight, height, body mass index), comorbidities data (e.g., diabetic status, hypertension, asthma), medical imaging data (e.g., MRI scans, CT scans, X-ray results), lifestyle indicators data (e.g., smoking status, alcohol consumption, exercise frequency, sleep patterns) and feedback (e.g., vocal responses, questionnaire answers, self-reported health, emotions, behavior). Such physiological data may be categorized into two types: sensor data and feedback associated with a subject. The sensor data may be collected from the one or more clusters of sensors and/or sensor electrodes associated with the one or more wearable assemblies, while the feedback may be collected through an interactive recording system (e.g., a microphone integrated with the one or more wearable assemblies or a graphical user interface) that may establish a communication channel between the subject and a computing system.

Based on the physiological data of the subject, a set of selection parameters may be configured to initiate the XCS. The set of selection parameters comprise waveform parameters (e.g., frequency, amplitude, pulse width, shape, waveform pattern etc.). In some aspects, the set of selection parameters comprising the waveform parameters may be configured, in addition to the initial stimulation amplitude associated with the invasive technique, by a medical professional to regulate the invasive technique according to the subject's needs or specific treatment requirements. In some aspects, the set of selection parameters may be configured based on rhythmic parameters (e.g., alignment with circadian rhythms, timing based on sleep-wake cycles, peak energy periods and/or subject-provided schedule preferences). In some aspects, the set of selection parameters may be configured based on event-based parameters (e.g., duration between stimulations according to therapeutic protocols, synchronization with other treatments, interventions and/or subject-provided schedule preferences). Based on the set of selection parameters, a stimulation generator may guide the delivery of the XCS to a cortical region of a subject.

Some aspects of the present disclosure relate to techniques to further adjust the set of selection parameters based on the feedback derived from the physiological data of a subject. In some aspects, the feedback may be an active feedback (e.g., answers to questionnaires, prompts or direct queries about the physical and mental state of the subject) or a passive feedback (e.g., recorded voice logs, tone of voice). Based on the feedback, the configuration of the set of selection parameters may be influenced by one or more of the following conditions: (1) if the subject is in recovery or remission; (2) if the subject is entering relapse; and (3) if the subject is in a steady state, as indicated by the feedback. The set of selection parameters may be adjusted—either increased, decreased or remain stable—based on the one or more of the conditions. Based on the adjusted set of selection parameters, the XCS may be regenerated by a stimulation generator and delivered to the cortical region of the subject via the stimulation electrode.

The XCS may include a single pulse, a series of pulses or a continuous waveform comprising sinusoidal waveform, square waveform, tonic stimulation, phasic stimulation, burst stimulation, ramp stimulation or cyclic stimulation. The XCS delivered, via a stimulation electrode, may be used to treat a range of neurological and psychiatric conditions including, but not limited to, movement disorders (e.g., Parkinson's disease, essential tremor or dystonia), mood disorders (e.g., depression and anxiety), obsessive-compulsive disorder (OCD), chronic pain, cognitive decline, and other conditions affecting brain function and motor control.

In some embodiments, a system is provided that includes one or more data processors and a non-transitory computer readable storage medium containing instruction which, when executed on the one or more data processors, cause the one or more data processors to perform part or all of one or more methods disclosed herein.

In some embodiments, a computer-program product tangibly embodied in a non-transitory machine-readable storage medium, including instructions configured to cause one or more data processors to perform part or all of one or more methods or processes disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

Various embodiments are described hereinafter with reference to figures. It should be noted that the figures are not drawn to scale and that the elements of similar structures or functions are represented by like reference numerals throughout the figures. It should also be noted that the figures are only intended to facilitate the description of the embodiments. They are not intended as an exhaustive description of the disclosure or as a limitation on the scope of the disclosure.

FIG. 1 illustrates an example overview of a process of calibrating a set of selection parameters associated with an invasive technique based on a non-invasive technique in accordance with some aspects of the present disclosure.

FIG. 2A shows an example illustration of a head-mounted device of one or more wearable assemblies in accordance with some aspects of the present disclosure.

FIG. 2B shows an example illustration of a wrist-worn device of the one or more wearable assemblies in accordance with some aspects of the present disclosure.

FIG. 3A shows an example illustration of normalized peaks of recruitment curves associated with the non-invasive technique in accordance with some aspects of the present disclosure.

FIG. 3B shows an example illustration of normalized peaks of recruitment curves associated with the invasive technique in accordance with some aspects of the present disclosure.

FIG. 4A illustrates a motor threshold by delivering a stimulus of the non-invasive technique in accordance with some aspects of the present disclosure.

FIG. 4B illustrates the recruitment curves associated with the non-invasive technique in accordance with some aspects of the present disclosure.

FIG. 4C illustrates the recruitment curves associated with the invasive technique in accordance with some aspects of the present disclosure.

FIG. 5 shows an example illustration of initiating a XCS therapy based on the set of selection parameters in accordance with some aspects of the present disclosure.

FIG. 6 shows an example illustration of an XCS-EEG module to administer the set of selection parameters based on the physiological data of the subject in accordance with some aspects of the present disclosure.

FIG. 7 shows an example illustration of a rhythmic dosing module to configure the set of selection parameters based on the physiological data of the subject in accordance with some aspects of the present disclosure.

FIG. 8 shows an example illustration of an event-based dosing module to configure the set of selection parameters based on the physiological data of the subject in accordance with some aspects of the present disclosure.

FIG. 9 shows an example illustration of a feedback module as part of the physiological data of the subject and to revise the set of selection parameters to initiate a regenerated XCS therapy in accordance with some aspects of the present disclosure.

FIG. 10 illustrates an exemplary workflow to initiate the XCS therapy to the subject based on the set of selection parameters in accordance with some aspects of the present disclosure.

FIG. 11 illustrates an exemplary workflow to calibrate the set of selection parameters associated with the invasive technique based on the non-invasive technique in accordance with some aspects of the present disclosure.

### DETAILED DESCRIPTION

Some embodiments of the present disclosure relate to techniques for delivering targeted XCS therapy tailored to a range of physiological conditions associated with the subject. The physiological conditions may include, but are not limited to, neurological disorders (e.g., Parkinson's disease, epilepsy, Alzheimer's disease), psychiatric disorders (e.g., obsessive-compulsive disorder (OCD), depression, anxiety), movement disorders (e.g., essential tremor, dystonia, ataxia), cognitive decline disorders (e.g., mild cognitive impairment, dementia), chronic pain (e.g., fibromyalgia, back pain, neuropathic pain), and other conditions affecting brain function and motor control.

Administrating stimulation therapies and determining a progression of physiological conditions may be complicated, in part, due to a variation in how subjects may respond to a XCS therapy, as well as the dynamic nature of certain conditions. For instance, factors such as age, genetics, comorbidities and/or lifestyle habits may influence how a subject's body reacts to a XCS therapy. Additionally, the progression of certain physiological conditions such as neurological or cardiovascular conditions may also fluctuate over time and require continuous monitoring and adjustment of the XCS therapy. The disclosed techniques may include initiating a XCS therapy (e.g., an externally powered cortical stimulation (XCS)) through an invasive technique by obtaining data from a non-invasive technique and/or physiological data (e.g., sensor data and feedback) associated with a subject.

The data obtained from the non-invasive technique may be used to calibrate a set of selection parameters associated with the invasive technique. The data may include determining a motor threshold based on a stimulus applied over a motor cortex region of a subject using the non-invasive technique. Based on the stimulus, a motor response (e.g., thumb or finger movement, hand grip or other voluntary movements of the body) of the subject may be identified in one or more regions controlled by the motor cortex. The motor response may determine the point at which the stimulus associated with the non-invasive technique may be strong enough to consistently evoke a motor response. The intensity of the stimulus that triggers a motor response in the identified region with a 50% probability may be defined as the motor threshold.

The non-invasive technique may further be employed over a cortical region, other than the motor cortex, to obtain additional data, which may include determining recruitment curves by sweeping stimulation amplitudes between 0% and 100% of the intensity associated with the non-invasive technique and recording evoked responses by using one or more electroencephalography (EEG) electrodes. In some aspects, the EEG electrodes may be integrated with one or more wearable assemblies. From the mapped recruitment curves, normalized peaks associated with the non-invasive technique at the motor threshold may be identified. The cortical region, other than the motor cortex, may include (for example) sensory cortex, visual cortex, auditory cortex, prefrontal cortex (e.g., left and right dorsolateral prefrontal cortex (DLPFC)) or language areas (e.g., Broca's and Wernicke's areas).

Based on the data from the non-invasive technique, an invasive technique may be employed that comprises implantation of a stimulation electrode over a cortical region of the subject depending on therapeutic requirements of a XCS therapy. In some aspects, the cortical region in which the stimulation electrode is implanted may correspond to, or overlap with, the cortical region targeted by the non-invasive technique. The placement of the stimulation electrode may be configured via an XCS implantation module. The XCS implantation module may require an invasive procedure (e.g., a burr hole procedure) to implant the stimulation electrode, ensuring that the stimulation electrode may be placed accurately to deliver the desired therapeutic effects. In some aspects, a stimulation electrode may be implanted before obtaining the data from the non-invasive technique.

The invasive technique may be applied, via the stimulation electrode, to obtain data including recruitment curves and normalized peaks of the recruitment curves based on stimulation amplitudes applied over the cortical region of the subject. The recruitment curves may be mapped by sweeping the simulation amplitudes between 0 and 15 mA of the intensity associated with the invasive technique and evoked responses may be recorded using the one or more EEG electrodes integrated in a wearable assembly of the one or more wearable assemblies. Based on the responses evoked, normalized peaks of the recruitment curves associated with the invasive technique at the motor threshold may be identified.

After obtaining data from the invasive technique, the set of selection parameters may be calibrated by matching the normalized peaks of the recruitment curves associated with both the non-invasive technique and the invasive technique. The matching process may identify the points of maximum response on the recruitment curves, which correspond to the most significant physiological reactions at the respective stimulation amplitudes. The stimulation amplitude that corresponds between the non-invasive and invasive techniques may be determined as an initial stimulation amplitude associated with the set of selection parameters for the invasive technique, which may subsequently be used to initiate an XCS to the cortical region of the subject.

In addition to the initial stimulation amplitude associated with the invasive technique, the disclosed techniques may further include setting the set of selection parameters by a trained professional according to the subject's needs or specific treatment requirements. The set of selection parameters may comprise waveform parameters including (for example) stimulation site, amplitude, intensity, frequency, pulse width, waveform type, and duration. The waveform parameters may be configured by an XCS-EEG module and transmitted to a parameter selection module. Based on the set of selection parameters (e.g., configured waveform parameters), the XCS may be generated via a stimulation generator.

In some aspects, the set of selection parameters may be configured using rhythmic parameters (e.g., alignment with circadian rhythms, timing based on sleep-wake cycles, peak energy periods and/or subject-provided schedule preferences), which may be determined by a rhythmic dosing module. In some aspects, the set of selection parameters may be configured using event-based parameters (e.g., a duration between stimulations according to therapeutic protocols, synchronization with other treatments, interventions and/or subject-provided schedule preferences), which may be determined by an event-based dosing module. In some aspects, the set of selection parameters (e.g., the rhythmic parameters and/or the event-based parameters) may influence the configuration of the waveform parameters configured by the XCS-EEG module.

The XCS may be initiated using the stimulation electrode that may be externally powered and digitally programmed via a magnetic coil associated with a wearable assembly of one or more wearable assemblies including (for example) a head-mounted device. Other one or more wearable assemblies include one or more wrist-worn devices, Holter monitors, electromyography (EMG) patches, silicon patches, pulse oximeters, blood pressure monitors, glucose monitors and smart monitors. Each wearable device of the one or more wearable devices may comprise of a data acquisition assembly having one or more clusters of sensors and/or sensor electrodes to collect the sensor's data, which may correspond to the physiological data of the subject. The sensor's data may include neural data (e.g., EEG, brain activity), cardiovascular data (e.g., heart rate, blood pressure, ECG), respiratory data (e.g., respiration rate, oxygen saturation), movement and activity data (e.g., accelerometer data, step count, gait analysis), environmental data (e.g., temperature, humidity, noise levels), medical imaging data (e.g., data from MRI scans, CT scans, X-ray) and/or lifestyle indicators data (e.g., exercise frequency, sleep patterns). The head-mounted device may wirelessly receive from other devices of the one or more wearable devices the physiological data of a subject.

Further, the feedback derived from the physiological data may be collected through an interactive system (herein after, referred to as an interaction module) integrated with one or more wearable assemblies including (for example) a head-mounted device or one or more wrist-worn devices. The interaction module may include a communication interface (e.g., microphone, speakers and/or a graphical user interface (GUI)) to facilitate the creation of a communication channel between the subject and a computing system. The feedback may include, but is not limited to, self-reported health data (e.g., subjective health status, pain levels), emotional and behavioral data (e.g., emotional states, stress levels, behavior assessments), vocal responses (e.g., speech patterns, tone of voice), questionnaire answers (e.g., responses to health or wellbeing surveys), lifestyle indicators data (e.g., smoking status, alcohol consumption) and/or demographic data (e.g., age, gender, race).

Based on the feedback derived from the physiological data of the subject and current waveform parameters, the set of selection parameters may be reconfigured and adjusted. The feedback from the subject may divided into an active feedback (e.g., answers to questionnaires, prompts or direct queries about the physical and mental state of the subject) or a passive feedback (e.g., recorded voice logs, tone of voice). Based on the feedback, mental and emotional state of the subject may be assessed and the configuration of the waveform parameters may be influenced by one or more of the following conditions: (1) if the subject is in recovery or remission, the waveform parameters may be reduced or stabilized; (2) if the subject is entering relapse, the waveform parameters may be increased; and (3) if the subject is in a steady state, no changes to the waveform parameters may be needed. The updated waveform parameters may become the set of selection parameters, which may be used to regenerate the XCS via the simulation generator. The stimulation electrode may initiate the regenerated XCS to the cortical region of the subject.

The XCS may include various forms of electrical signals including a single pulse, a series of pulses or a continuous waveform. The continuous waveforms may comprise tonic stimulation (sustained pulses over time), phasic stimulation (intermittent pulses), frequency-modulated pulses (where frequency varies over time) or amplitude-modulated pulses (where the intensity of pulses changes). The XCS may also be delivered in specific patterns including burst stimulation (multiple pulses in rapid succession) or rhythmic pulses at varying frequencies. Such forms of XCS may be tailored to the specific physiological conditions (e.g., neurological disorders, chronic pain etc.) and the therapeutic needs of the subject. The XCS may influence neuronal activity, either by enhancing or inhibiting brain functions, depending on the set of selection parameters, as disclosed herein.

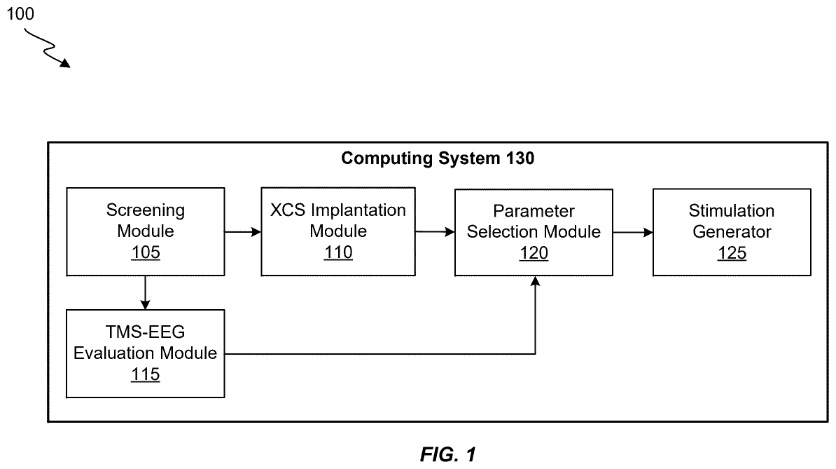

FIG. 1 illustrates an example overview of a process 100 of calibrating a set of selection parameters associated with an invasive technique based on a non-invasive technique in accordance with some aspects of the present disclosure. The process 100 calibrates the set of selection parameters associated with the invasive technique (e.g., an externally powered cortical stimulation (XCS)) that may be configured by a parameter selection module 120. The process 100 begins by receiving the physiological data of the subject that may then be accessed by a screening module 105. The screening module 105 may perform an initial assessment to evaluate the subject's physiological conditions and determine if such conditions may indicate a potential risk associated with a psychiatric disorder.

The initial assessment is based on the physiological data of the subject, which may be obtained from the one or more wearable assemblies. Each wearable assembly may comprise a data acquisition assembly having one or more clusters of sensor and/or sensor electrodes. The one or more clusters of sensor and/or sensor electrodes may include electroencephalography (EEG) electrodes, electrocardiography (ECG) electrodes, electromyography (EMG) electrodes, electrooculography (EOG) electrodes, photoplethysmography (PPG) sensors, temperature sensors or near-infrared spectroscopy (NIRS) sensors. Such clusters may capture a range of physiological signals including muscle activity, eye movement, heart rate, blood oxygen levels and skin temperature of the subject. Additionally, the physiological data may also be obtained from medical imaging devices (e.g., MRI, fMRI, PET, TMS) and diagnostic tools (e.g., blood tests, CSF analysis, genetic testing etc.), physiological and cognitive assessments (e.g., BDI, HDRS, MINI, MADRA) and feedback from the subject.

The collected physiological data may indicate physiological conditions including, but not limited to, neurological disorders (e.g., Parkinson's disease, epilepsy, Alzheimer's disease), psychiatric disorders (e.g., obsessive-compulsive disorder (OCD), depression, anxiety), chronic pain (e.g., fibromyalgia, back pain, neuropathic pain), movement disorders (e.g., essential tremor, dystonia, ataxia), and cognitive decline (e.g., mild cognitive impairment, dementia), as well as other conditions affecting the physical or mental state of the subject.

Based on the initial assessment performed by the screening module 105, the physiological data may further be processed by an XCS implantation module 110 and a TMS-EEG evaluation module 115. For instance, if a psychiatric disorder is detected, the process 100 may require employing the invasive technique, which may comprise the implantation of a stimulation electrode through the XCS implantation module 110 to further deliver the XCS using the stimulation electrode. However, before implantation, a non-invasive technique may be used to assess evoked responses to a targeted stimulus. In some aspects, the non-invasive technique may comprise transcranial magnetic stimulation (TMS) delivered using TMS coils, with the evoked responses to a targeted magnetic pulse being assessed through the TMS-EEG evaluation module 115. Other non-invasive technique may include, but is not limited to, transcranial direct current stimulation (tDCS), transcranial ultrasound stimulation (TUS), transcranial focused ultrasound (FUS), functional near-infrared spectroscopy (fNIRS) or magnetic seizure therapy (MST).

In the present disclosure, TMS may be applied over cortical regions of the subject through the TMS coils and the evoked responses may be recorded by wearable sensor electrodes (e.g. EEG electrodes). In some aspects, the sensor electrodes may be integrated within a wearable assembly of the one or more wearable assemblies that may record the evoked responses. The recorded responses may be processed by the TMS-EEG evaluation module 115, which may filter and transmit data associated with the non-invasive technique to the parameter selection module 120.

After obtaining data from the TMS-EEG evaluation module, the XCS implantation module 110 may employ an invasive technique by implanting a stimulation electrode. The XCS implantation module 110 may have to select an appropriate cortical region for the electrode placement based on therapeutic requirements of a subject and the intended outcomes of the XCS. In some aspects, the cortical region in which the stimulation electrode is implanted may correspond to, or overlap with, the cortical region targeted by the non-invasive technique. Presurgical imaging techniques, such as MRI or functional MRI (fMRI), may be conducted to precisely identify the cortical region. Once the cortical region is identified, the stimulation electrode may be surgically implanted into the target region through a burr hole procedure. In the burr hole procedure, a small hole may be drilled into the skull of the subject, typically in a location that provides a direct access to the cortical region of the brain. Following the insertion of the stimulation electrode, the burr hole is closed, and the implantation location may be monitored for proper placement through post-surgical imaging such as a CT scan. Once the recovery phase is complete, the stimulation electrode associated with the invasive technique may become handy for ongoing monitoring and stimulation with different adjustments made as necessary based on the physiological data of the subject.

The TMS-EEG evaluation module 115 may complement the XCS implantation module 110 by providing initial insights into the brain activity of the subject. The data associated with the non-invasive technique obtained from the TMS-EEG evaluation module 115 may be used to configure an initial stimulation amplitude for the invasive technique (e.g., the XCS). The parameter selection module 120 may process the initial stimulation amplitude as part of the set of selection parameters, based on which the XCS may be initiated. The parameter selection module 120 may further refine the set of selection parameters based on the therapeutic requirements of the subject. The set of selection parameters may then be provided to the stimulation generator 125 that may configure the XCS in accordance with the set of selection parameters. After configuration, the XCS may be initiated via the stimulation electrode to the cortical region of the subject.

The process 100 may be performed, in part, by a computing system 130 that operates in conjunction with other modules or hardware assemblies including the one or more wearable assemblies, medical imaging devices (e.g., devices associated with MRI, fMRI, CT scan), diagnostic equipment, environmental monitoring systems, surgical procedures or the like. The computing system 130 may be operated automatically, by the subject, a clinician or a medical provider associated with treating the subject, or an entity facilitating medical monitoring or treatment for the subject. The computing system 130 may include one or more wearable assemblies (e.g., a head-mounted device, a wrist-worn device), a mobile device (e.g., a smartphone), personal digital/data assistants (PDA), a tablet, a laptop, a desktop computer, a computer server etc. The computing system 130 may enable real-time communication between the subject and the computing system 130, facilitating updates and adjustments regarding the XCS therapy. The physiological data obtained by the one or more wearable devices and designated medical imaging devices are communicated to the computing system 130 over a network. The network may be a communication infrastructure that may facilitate the transfer of physiological signals, brain activity, and neural responses between the clinician's or medical provider's interface and the computing system 130. The network may include communication protocols such as wired connections (e.g., Ethernet, USB, or fiber optics) or wireless connections (e.g., Wi-Fi, Bluetooth, or cellular networks).

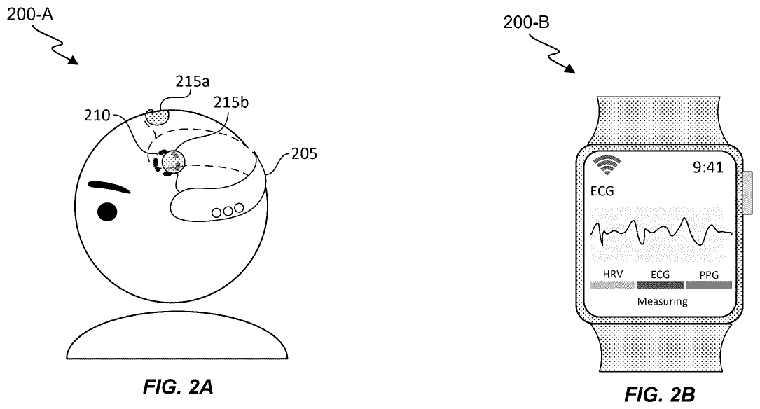

FIG. 2A shows an example illustration of a head-mounted device 205 of the one or more wearable assemblies in accordance with some aspects of the present disclosure. The one or more wearable assemblies may represent a sophisticated system for continuous and real-time monitoring of the physiological data of the subject. The head-mounted device 205 of the one or more wearable assemblies may comprise a data acquisition assembly having one or more clusters of sensor and/or sensor electrodes to obtain the physiological data of the subject.

According to the example illustration, the head-mounted device 205 of the one or more wearable devices includes sensor electrodes 215a and 215b placed on the scalp of the subject using an electrode lead. The sensor electrodes 215a-b may be EEG electrodes used to monitor the electrical activity of the brain, typically in response to external stimuli. The sensor electrode 215a-b may include an active electrode and a reference electrode. Both active and reference electrodes can be placed close to each other but are not electrically connected. The active electrode (e.g., the sensor electrode 215b) may be positioned near the target region on the scalp to detect the brain's electrical signals generated by neuronal activity. For instance, when the XCS therapy may be provided via a stimulation electrode 210, the active electrode may capture any changes in the brain's activity resulting from such stimulation. The reference electrode (e.g., the sensor electrode 215a) may provide a stable baseline signal against which the brain activity, recorded by the active electrode, can be compared. The differential configuration of the active and reference electrodes may allow for the assessment of changes in brainwaves or neural responses by measuring the difference in electrical potential between the two sensor electrodes. In some aspects, the sensor electrodes 215a-b may record responses evoked by non-invasive technique (e.g., TMS) and the invasive technique (e.g., XCS).

In some aspects, the head-mounted device 205 may support one or more clusters of sensors and/or sensor electrodes in addition to the senor electrode 205a-b. In one composition, the senor electrodes may integrate a range of physiological sensor electrodes such as EEG, EOG, EMG, and MEG that may be strategically placed on various parts of the skull to capture a comprehensive set of physiological data. The redundancy provided by multiple sensor electrodes may enhance the reliability for detecting subtle physiological changes that may be missed by a single sensor electrode. For example, one sensor electrode may focus on monitoring neural signals (e.g., EEG, MEG), while another may track eye movements (EOG) and muscle activity (EMG). Each sensor electrode of the one or more clusters of sensor electrodes may operate independently yet communicate with the computing system 130, which may enable continuous monitoring even if one sensor electrode experiences a temporary failure or interference.

Additionally, the head-mounted device 205 may comprise a magnetic coil that may externally power and digitally program the stimulation electrode 210 to significantly reduce the invasiveness of the XCS therapy. By externally powering the stimulation electrode 210, the need for invasive wiring or physical connectors may be eliminated that makes the XCS therapy comfortable for the subject. The head-mounted device 205 may ensure effective treatment without the need for implanted power sources or complex setups. Such a minimally invasive approach may enhance the experience of the subject while still providing precise control over the XCS therapy.

Furthermore, the head-mounted device 205 may integrate an interaction module that may facilitate bidirectional communication between the head-mounted device 205 and the subject. The bidirectional communication may be recorded in the form of voice logs, which may be used for further analysis or decision-making. Additionally, the interaction module may include a graphical user interface (GUI) that may visually interact with the subject to provide real-time feedback to the head-mounted device or the computing system 130. The configurations of the head-mounted device 205 may also be modified based on the physiological data of the subject that can be received wirelessly from an assembly of the one or more wearable assemblies or some preferences given by the subject.

FIG. 2B shows an example illustration of a wrist-worn device 200-B of the one or more wearable assemblies in accordance with some aspects of the present disclosure. The wrist-worn device 200-B may be equipped with the one or more clusters of sensors to continuously monitor the physiological data of the subject in real time. The sensors embedded within the wrist-worn device 200-B may include, but are not limited to, heart rate sensors to monitor cardiovascular activities, electrodermal sensors to track skin conductance and stress levels, temperature sensors to record body temperature, motion sensors (e.g., accelerometers and gyroscopes) to detect physical activity and movement patterns, blood oxygen sensors to measure oxygen saturation (SpO2) levels, and ECG sensors to monitor heart rhythm and its electrical activities. The physiological data collected by the one or more clusters of sensors may be transmitted wirelessly to the head-mounted device 205 or the computing system 130, which may adjust the XCS therapy based on real-time changes in the physiological data of the subject.

In addition to physiological monitoring, the wrist-worn device 200-B may also integrate the interaction module to continuously record voice logs of the subject that may provide insights into the subject's mental state. The wrist-worn device 200-B may also occasionally present a questionnaire to the subject via a GUI that may predict the mental state of the subject based on the subject's responses. The voice logs and questionnaires may be used by the computing system 130 for further analysis, assisting in decisions about the XCS therapy to be delivered to the subject. In some aspects, the interaction module of the wrist-worn device 200-B may notify the subject when to wear the head-mounted device 205 for administering the XCS therapy. Such notifications may ensure that the subject receives timely and appropriate treatment based on the subject's current physiological and mental condition.

In addition to the example illustrations of the one or more wearable assemblies 200-A and 200-B, other one or more wearable assemblies may include devices such as Holter monitors, ECG patches, and portable ECG devices like KardiaMobile and AliveCor for heart monitoring. Other devices may include pulse oximeters (e.g., Masimo MightySat and Withings Pulse Oximeter) for measuring oxygen saturation (SpO2) and pulse rate, wearable blood pressure monitors (e.g., Omron Evolv and QardioArm) for tracking blood pressure, and wearable glucose monitors (e.g., Dexcom G6 and Freestyle Libre) for continuous blood glucose monitoring. Additionally, smart scales (e.g., Withings Body+and Fitbit Aria) may be used to track weight, BMI, and body composition of the subject. However, the selection of wearable assemblies is not intended to limit the scope of the disclosure.

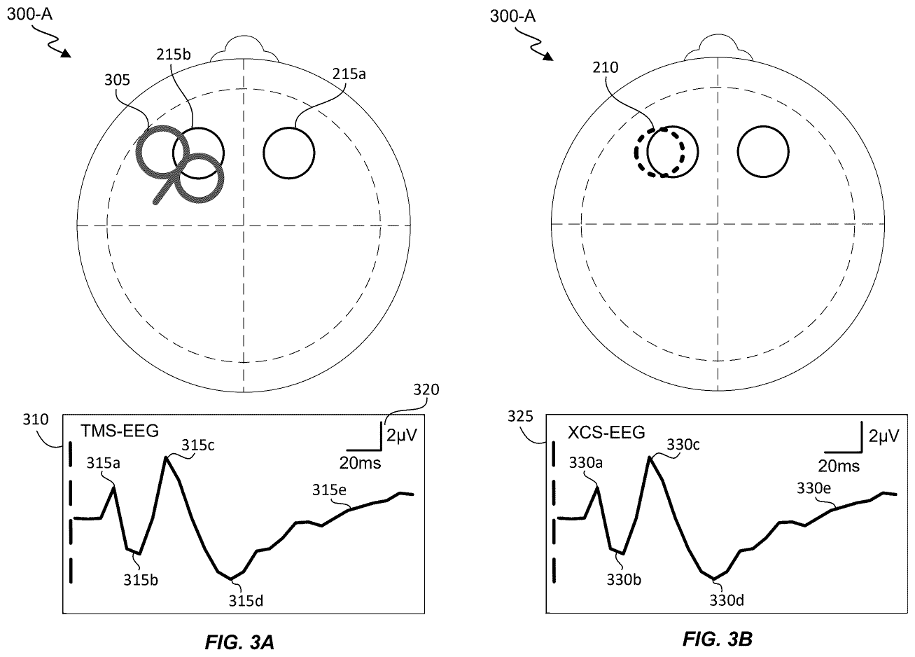

FIG. 3A shows an example illustration 300-A of normalized peaks of recruitment curves associated with the non-invasive technique (e.g., the TMS) in accordance with some aspects of the present disclosure. TMS is a non-invasive neuromodulation technique that may use electromagnetic fields to stimulate nerve cells in the brain or target cortical region. The TMS coils 305 may generate brief magnetic pulses that may induce small electrical currents in different regions, modulating neural activity in the subject. TMS may often be used in clinical and research settings for therapeutic purposes such as treating depression or investigating brain function by stimulating cortical regions including motor cortex or dorsolateral prefrontal cortex (DLPFC).

The normalized peaks of the recruitment curves obtained from evoked response generated by the TMS coils 305 (herein, after referred to as TMS-EEG response 310) may be identified and configured by the TMS-EEG evaluation module 115, as illustrated in FIG. 1. TMS by the TMS coils 305 may initially evaluate motor thresholds and neural response patterns (e.g., recruitment curves), which may provide a reference for configuring the initial stimulation amplitude associated with the set of selection parameters. According to the example illustration 300-A, the TMS coils 305 may initially be placed over hand knob of motor cortex to determine a motor threshold via EEG electrodes. The motor threshold may be a minimum stimulator output that elicits a motor response in the thumb with a 50% probability. To find the motor threshold, a single pulse TMS may be administered using the TMS coils 305. Once the motor threshold is set, the TMS coils 305 may be repositioned to the left DLPFC using a Beam-F3 technique that may ensure a precise alignment of the TMS coils 305. The Beam-F3 technique may help to achieve favorable stimulation for therapeutic or diagnostic purposes.

The TMS amplitudes may be swept between 0% and 100% of intensity associated with the TMS and evoked responses may be recorded by the EEG electrodes. The evoked responses may be configured to identify the TMS-EEG response 310 that may feature TMS-induced normalized peaks 315a-e, each representing different components of the evoked responses of the recruitment curves associated with the TMS (likely corresponding to a post-stimulation specific brain activity at different time intervals). Each peak within the TMS-induced normalized peaks 315a-e may demonstrate specific points in time and amplitude. Scale 320 of the TMS-EEG response 310 may represent a vertical scale of 2 μV demonstrating the amplitude of the EEG signal, while a horizontal scale of 20 ms may demonstrate the time after administering the stimulation by the TMS coils 305.

FIG. 3B shows an example illustration 300-B of normalized peaks of recruitment curves associated with the invasive technique (e.g., the XCS) in accordance with some aspects of the present disclosure. After the initial evaluation by the TMS-EEG evaluation module 115, the stimulation electrode 210 may be implanted over the left DLPFC by the XCS implantation module 110, as illustrated in FIG. 1. The stimulation electrode 210 may be activated and the stimulation amplitude may be varied, while the evoked responses are recorded by the EEG electrodes. The evoked responses, followed by an initial XCS by the stimulation electrode 210, may be configured to generate an XCS-EEG response 325 that may feature XCS-induced normalized peaks 330a-e, where each peak of the XCS-induced normalized peaks 330a-e of the recruitment curves may closely follow the pattern of peaks in the TMS-EEG response 310.

Each peak within the XCS-induced normalized peaks 330a-e may demonstrate specific points in time and amplitude based on the stimulation provided by the stimulation electrode 210. The XCS-EEG response 325 may be plotted on the same scale as the TMS-EEG response 310, where the vertical scale of 2 μV demonstrates the amplitude of the EEG signal and the horizontal scale of 20 ms may demonstrate the time after the stimulation by the stimulation electrode 210. The normalized peaks of the TMS-EEG response 310 and the XCS-EEG response 325 may be matched to calibrate the set of selection parameters for the XCS.

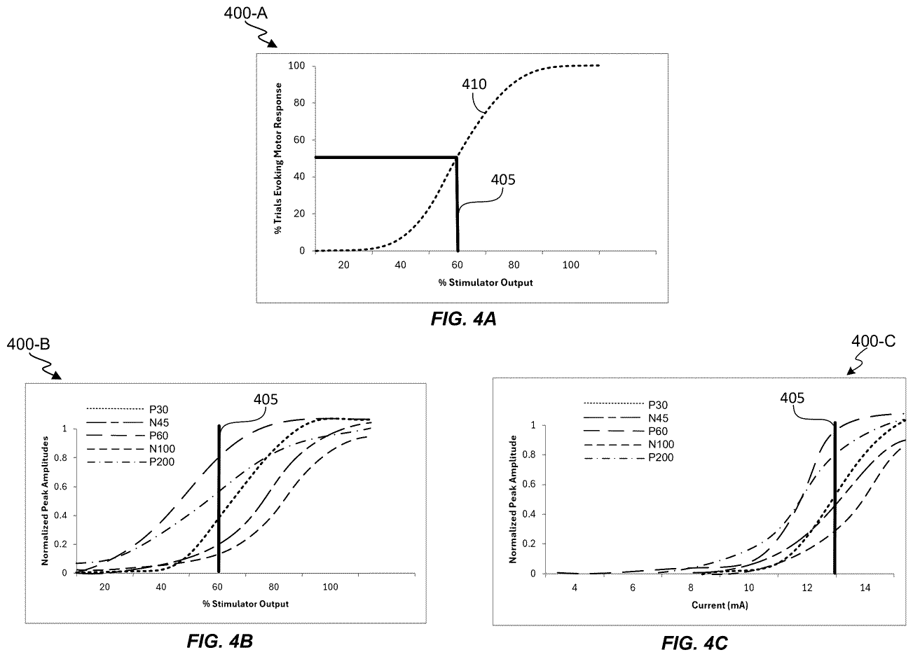

FIG. 4A illustrates the motor threshold 405 by varying stimulation amplitudes of the non-invasive technique (e.g., the TMS through TMS coils 305) in accordance with some aspects of the present disclosure. The TMS may be administered over the hand knob of a motor cortex to configure the motor threshold, as disclosed in FIG. 3A. The hand knob is known for controlling hand and thumb movements. The motor threshold 405 may be defined as the TMS intensity level that has a 50% probability of evoking a motor response (such as a thumb movement). The motor threshold 405 may form a baseline for the subject's responsiveness to TMS in the motor cortex. According to the illustration in FIG. 4A, a sigmoid curve 410 may represent the relationship between the stimulator's output percentage and the probability of a motor response. As the percentage of stimulator output increases, the probability of eliciting the motor response (such as a thumb movement) may also increase in a gradual, but S-shaped curve. The sigmoid curve 410 is typical of a dose-response relationship, where small increases in stimulation may lead to little or no response, but beyond a certain threshold, the likelihood of a response rises exponentially. The motor threshold 405 may be used as a reference point to ensure that the stimulation intensity is set to an appropriate level for inducing a consistent and measurable motor response.

FIG. 4B illustrates the recruitment curves associated with the non-invasive technique 400-B (e.g., the TMS) in accordance with some aspects of the present disclosure. After determining the motor threshold 405, the TMS coils 305 may be moved to left DLPFC, as disclosed in FIG. 3A. The TMS amplitudes may again be swept between 0% and 100%, and evoked brain responses may be recorded using the EEG electrodes. The recruitment curves may illustrate the relationship between the TMS intensity and the amplitude of the EEG response. Different recruitment curves (e.g., P30, N45, P60, N100, P200) may represent various evoked response components (e.g., positive and negative peaks) recorded during EEG and illustrated as the TMS-induced normalized peaks 315a-e in the TMS-EEG response 310. The positive and negative peaks may indicate whether the EEG signal may move in a positive direction or a negative direction. For instance, the recruitment curve P30 means a positive peak occurring at approximately 30 milliseconds after stimulation, the recruitment curve P60 is a positive peak at 60 milliseconds, and so on. The peaks of the recruitment curve (P30, N45, P60) may reflect early cortical responses to stimulation (mapped as 315a-c, as illustrated in the TMS-EEG response 310) and often represent the brain's initial, fast reactions, possibly related to sensory processing or a direct motor activation. The peaks (N100, P200) may occur later (mapped as 315d-e, as illustrated in the TMS-EEG response 310) and may typically be associated with cognitive processing or more complex neural interactions. Recruitment curves N100 and P200 may often be used to study and analyze attention, perception and decision-making processes.

The vertical line may represent the motor threshold 405 of TMS, which may illustrate how evoked responses at DLPFC relate to the motor threshold's stimulator output. The normalized amplitudes of the TMS-induced normalized peaks 315a-e may be identified, based on the recruitment curves associated with the TMS, to establish a specific reference for the subject's neural activity when stimulated in the DLPFC.

FIG. 4C illustrates recruitment curves associated with the invasive technique 400-C (e.g., the XCS) in accordance with some aspects of the present disclosure. The stimulation amplitudes associated with the XCS delivered via the stimulation electrode 210 may be varied between 0 and 15 mA, and evoked responses are recorded by the EEG electrodes. The recruitment curves associated with the XCS may be similar to that of the recruitment curves associated with TMS. The target is to map the recruitment curves for XCS and find the initial stimulation amplitude that may generate evoked responses similar to those that are generated by the TMS, as disclosed in FIG. 4B. Based on the recruitment curves associated with the XCS, the XCS-induced normalized peaks 330a-e may be identified.

The example illustrations of FIGS. 4A-C may show how the recruitment curves may be determined by sweeping the stimulation amplitudes of the TMS (e.g., non-invasive technique) and the XCS (e.g., invasive technique). Based on the recruitment curves, normalized peaks (e.g., TMS-induced normalized peaks 315a-e and XCS-induced normalized peaks 330a-e) may be identified. After obtaining the data from the non-invasive technique (including, motor threshold 405, recruitment curves associated with the non-invasive technique 400-B and TMS-induced normalized peaks 315a-e) and the invasive technique (including recruitment curves associated with the invasive technique 400-C and XCS-induced normalized peaks 330a-e), the set of selection parameters may be calibrated by matching the TMS-induced normalized peaks 315a-e and the XCS-induced normalized peaks 330a-e. based on the matched normalized peaks, an initial stimulation amplitude may be configured for the XCS that may be initiated by the stimulation electrode 210.

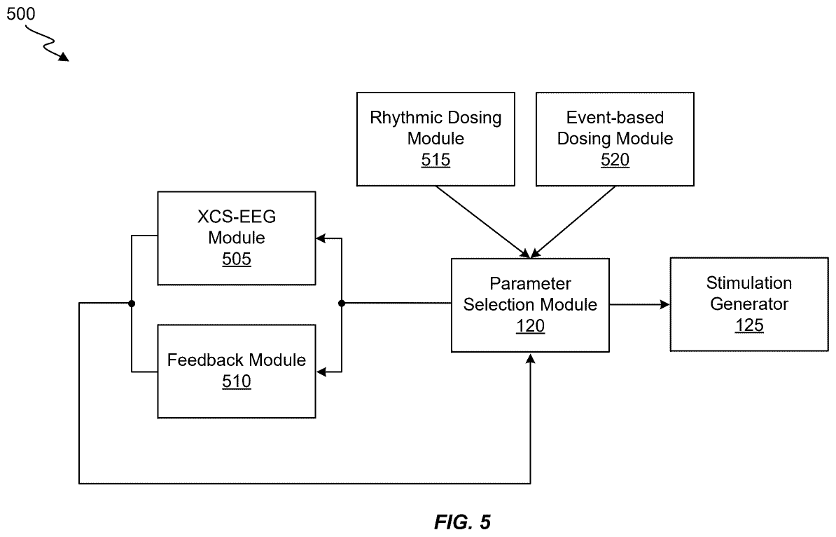

FIG. 5 shows an example illustration 500 for initiating the XCS based on the set of selection parameters in accordance with some aspects of the present disclosure. The example illustration 400 may administer the XCS based on the set of selection parameters configured by the parameter selection module 120, in addition to configuration of the initial stimulation amplitude for the XCS. The XCS may be delivered based on the configuration of the set of selection parameters comprising waveform parameters. The waveform parameters may be configured by an XCS-EEG module 505. In some aspects, the waveform parameters configured by the XCS-EEG module 505 may be set or adjusted by a medical provider or a trained professional in the field, which may ensure that the XCS therapy is tailored to the specific needs of the subject and aligned with therapeutic goals.

The XCS-EEG module 505 may pertain to the physical characteristics of the XCS provided by the stimulation electrode 210. The XCS-EEG module 505 may define the waveform parameters associated with the XCS, where the waveform parameters may include characteristics such as frequency, amplitude, pulse width, shape and/or waveform pattern. Such characteristics may influence the effectiveness of the XCS therapy, as the XCS-EEG module 505 may determine how the XCS may interact with the electrical activities of the brain. The waveform parameters from the XCS-EEG module 505 may be transmitted to the parameter selection module 120. The parameter selection module 120 may configure the waveform parameters as part of the set of selection parameters. Based on this configuration, the set of selection parameters may be used to generate the XCS using the stimulation generator 125, which may be delivered to the cortical region of the subject.

In some aspects, the set of selection parameters may be configured by rhythmic parameters, which may be determined by a rhythmic dosing module 515. The rhythmic dosing module 515 may align the XCS with the natural biological rhythms of the subject. The rhythmic dosing module 515 may ensure that the set of selection parameters selected to deliver the XCS may synchronize with the body's ongoing physiological processes. The rhythmic parameters configured by the rhythmic dosing module 515 may be based on neural rhythms (e.g., alpha, beta, delta waves), cardiac rhythms (e.g., heart rate), respiratory rhythms (e.g., the pattern of inhalation and exhalation), circadian rhythms (e.g., the 24-hour sleep-wake cycle), ultradian rhythms (e.g., REM sleep cycles, heart rate variability), infradian rhythms (e.g., menstrual cycle, seasonal rhythms) and/or circannual rhythms (e.g., experiencing seasonal affective disorder (SAD) in winter, where subjects may feel more depressed due to reduced sunlight exposure, and feeling more energetic or happy during the summer months when sunlight is abundant).

In some aspects, the rhythmic parameters may be configured based on a specific time of day for initiating the XCS, following rhythms that adhere to fixed cycles (e.g., regular circadian rhythms). The regular rhythms may include circadian rhythms, ultradian rhythms, infradian rhythms and circannual rhythms. By aligning the XCS with such natural biological rhythms, the set of selection parameters may be configured for the subject's inherent physiological processes.

In some aspects, the set of selection parameters may be configured by event-based parameters, which may be determined by an event-based dosing module 520. The event-based parameters may include synchronization of the XCS with other ongoing treatments or therapies including pharmaceuticals, cognitive behavioral therapy, meditation, or other neuromodulation therapies (e.g., deep brain stimulation, vagus nerve stimulation, transcranial magnetic stimulation, electroconvulsive therapy, repetitive transcranial magnetic stimulation, focused ultrasound, spinal cord stimulation or the like). Additionally, the XCS may be coordinated with physical therapies, biofeedback, mindfulness practices, acupuncture, and other alternative or complementary treatments. At the administration of such therapies, the subject may opt to activate the XCS that may ensure that the XCS is delivered in conjunction with the other treatments.

The rhythmic parameters and/or event-based parameters may influence the set of selection parameters comprising waveform parameters (e.g., frequency, amplitude, pulse width, and waveform pattern). Once the parameters are configured, the set of selection parameters may be processed by the parameter selection module 120 and then sent to the XCS-EEG module 505, where the characteristics of the XCS may be fine-tuned. The waveform parameters may be verified and finalized by the parameter selection module 120 before being sent to the stimulator generator 125, which may generate the XCS to be delivered to the cortical region of the subject. In some aspects, other modules or aspects, in addition to those described herein, may also contribute to the configuration and delivery of the XCS therapy.

After the XCS may be delivered to the cortical region of the subject, the set of selection parameters may be adjusted according to feedback from the feedback module 510 associated with the physiological data of the subject. The feedback from the subject may include an active feedback (e.g., answers to questionnaires, prompts or direct queries about the physical and mental state of the subject) or a passive feedback (e.g., recorded voice logs, tone of voice). The computing system 130, after receiving the feedback, may process the feedback to predict the mental state of the subject. The prediction or the decision from the feedback module 510 may be transmitted to the XCS-EEG module 505 to readjust the waveform parameters. Readjusted parameters may be fed back to the parameter selection module 120, ultimately guiding the delivery of regenerated XCS via the stimulation generator 125.

In some aspects, the XCS and the regenerated XCS may be similar. For instance, if the subject's mental state is stable, the same stimulations may be repeated to maintain the steady-state condition. In some other aspects, the XCS and the regenerated XCS may differ. For instance, if the subject is recovering or entering a relapse, the regenerated XCS may be adjusted—either increased or decreased—based on the mental state of the subject. Such adjustments may be made in response to the feedback, which ensures that the XCS therapy is tailored to the subject's current mental or physiological state.

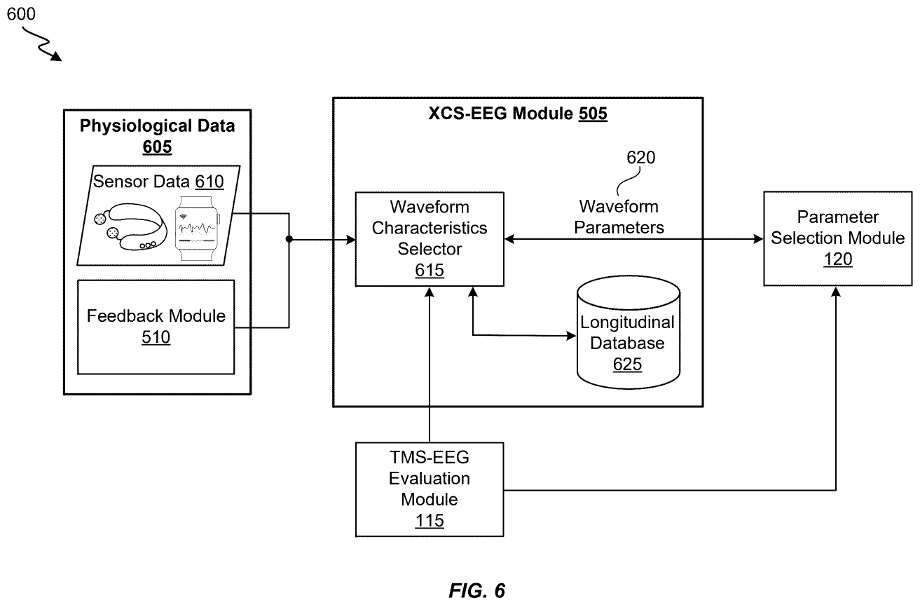

FIG. 6 shows an example illustration of an XCS-EEG module 505 to administer the set of selection parameters based on physiological data 605 of the subject in accordance with some aspects of the present disclosure. The XCS-EEG module 505 may be responsible for configuring and managing the characteristics of each XCS delivered by the stimulation electrode 210 to administer the XCS therapy. The XCS-EEG module 505 may receive its inputs from the physiological data 605 associated with the subject, the parameter selection module 120 or the TMS-EEG evaluation module 115.

The physiological data 605 may refer to a broad range of biological signals that reflect the physical and mental state of the subject. The physiological data may comprise of sensor data 610 and the data from the feedback module 510, obtained from the one or more wearable assemblies. The sensor data 610 comprises neural data (e.g., brain function, brain-system dysfunction), cardiovascular data (e.g., heart rate variability, ECG, pulse rate, oxygen saturation), respiratory data (e.g., respiratory rate, breathing patterns), movement and activity data (e.g., physical activity levels, gait patterns), environmental data (e.g., light exposure, ambient sound levels), demographic data (e.g., age, gender, race), anthropometric data (e.g., weight, height, body mass index), comorbidities data (e.g., diabetic status, hypertension, asthma), medical imaging data (e.g., MRI scans, CT scans, X-ray results), and lifestyle indicators data (e.g., smoking status, alcohol consumption, exercise frequency, sleep patterns). Additionally, the feedback (e.g., vocal responses, questionnaire answers, self-reported data on health, emotions and behavior, lifestyle indicators data, demographic data) from the feedback module 510 may also be part of the physiological data 605 of the subject. The feedback module 510 may predict the mental state of the subject based on the interaction of the subject with the one or more wearable assemblies. The feedback module 510 also incorporates decisions made by the subject, which are then used to process the set of selection parameters, thereby contributing to the overall adaptive response of the computing system 130.

In addition to the physiological data, the XCS-EEG module 505 may receive input from the parameter selection module 120. The input from the parameter selection module 120 may include parameters configured by the rhythmic dosing module 515, the event-based dosing module 520 and/or the feedback module 510, which may then be used to readjust the waveform parameters 620 associated with each stimulation of the XCS. An input may also be received from the TMS-EEG evaluation module 115 that may assist in selecting the initial stimulation amplitude associated with the set of selection parameters for the XCS therapy, as illustrated in FIGS. 3A-4C.

The XCS-EEG module 505 includes a waveform characteristics selector 615 and longitudinal database 625. The waveform characteristics selector 615 may configure or readjust the characteristics (e.g., frequency, amplitude, pulse width, shape and/or waveform pattern) of each stimulation of the XCS therapy (e.g., the XCS and the regenerated XCS). The frequency of the XCS may refer to the rate at which the stimulation pulses are delivered over time. The frequency may determine the rhythm or pacing of each stimulation of the XCS therapy, which may influence the neural response significantly. Higher frequencies may produce different effects than lower frequencies that may potentially enhance or modulate the therapeutic impact. The amplitude of the XCS may denote the strength or intensity of the electrical pulses, which may control how much energy may be delivered to the target tissue. A higher amplitude may result in a stronger stimulation that may be needed for deeper or more intense therapeutic effects, while lower amplitudes may generally be used for more subtle interventions. The pulse width of the XCS may refer to the duration of each individual pulse or stimulation within the XCS therapy. A longer pulse width may supply more energy per pulse that potentially influences the depth and efficacy of the XCS therapy, whereas a shorter pulse width may result in quicker and more transient interactions with the target tissue. The shape may refer to the overall shape of the XCS (e.g., square, sine or triangular). Different shapes may be picked depending on the specific needs of the XCS therapy, as each type may have distinct effects on the neural circuits. In some other aspects, one or more characteristics may be modified based on the physiological data 605 of the subject to raise the therapeutic effects.

The longitudinal database 625 may also be utilized by the waveform characteristics selector 615 to refine the configuration of each stimulation of the XCS therapy. The longitudinal database 625 may manage detailed logs of prior stimulation therapies and/or document the characteristics of the waveform parameters 620 that may have been finalized for each session. The longitudinal database 625 may also store the initial stimulation amplitude or initial parameters associated with the set of selection parameters, derived from the TMS-EEG evaluation module 115, to track changes. The waveform parameters 620, currently configured, may be logged continuously in the longitudinal database 625 for a reference. Furthermore, the longitudinal database 625 may track the subject's physiological changes over time, which may offer insights into how the subject's health condition evolves in response to the XCS therapy. By maintaining a historical record of such interactions and adjustments, the longitudinal database 625 may ensure that future stimulations are better aligned with the current needs of the subject. The longitudinal database 625 may be housed either inside or outside the XCS-EEG module 505, however, the location of the longitudinal database is not intended to limit the scope of the disclosure.

In some aspects, the longitudinal database 625 may be implemented as a cloud-based database, which may serve as a centralized and accessible storage solution. Such cloud-based database may be a single, unified database used by the computing system 130, which may allow seamless access and synchronization of data across multiple modules within the computing system 130, regardless of their physical location. Other possible database implementations may include, but are not limited to, relational databases (SQL), NoSQL databases, in-memory databases, distributed databases or hybrid databases etc., each offering features that are well suited for different use cases.

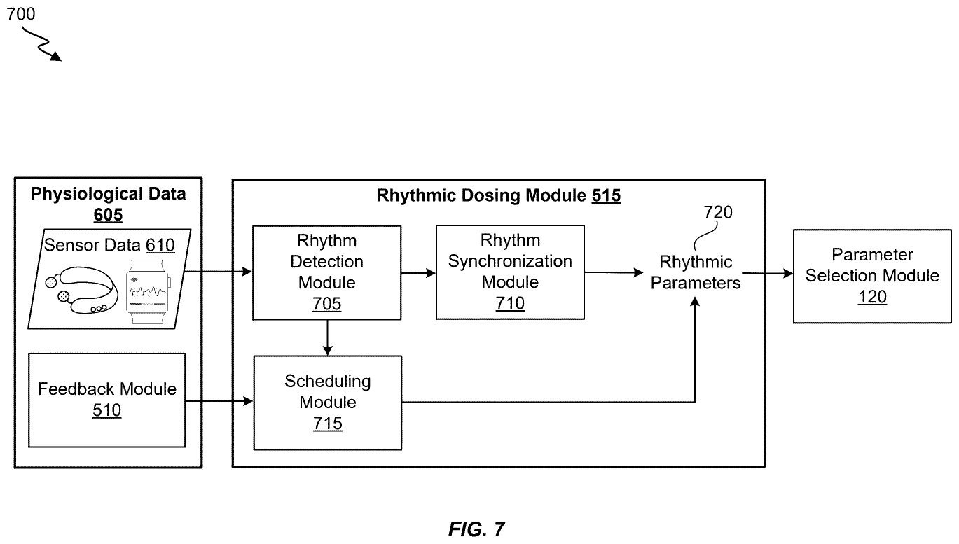

FIG. 7 shows an example illustration of a rhythmic dosing module 515 to configure a set of selection parameters based on the physiological data 605 of the subject in accordance with some aspects of the present disclosure. The rhythmic dosing module 515 may configure the rhythmic parameters 720 that may tailor the timing and delivery of the XCS to align with the subject's natural biological rhythms. The rhythmic dosing module 515 may employ a rhythmic detection module 705 to identify the natural biological rhythms of the subject, utilizing the physiological data 605 collected from the one or more wearable assemblies.

Based on the physiological data 605 of the subject, the rhythmic detection module 705 may detect the subject's inherent rhythm patterns and categorizes them into regular rhythms and flowing rhythms. The regular rhythms, also referred to as fixed rhythms, are predictable and may follow consistent cycles (e.g., sleep-wake cycle, hormone secretion and/or body temperature). The regular rhythms (e.g., circadian, ultradian and infradian rhythms) may repeat at predictable intervals and the subject may synchronize the XCS with such rhythms. For instance, if subjects know that their energy peaks occur in the morning or if they prefer therapy before sleep, they can select these times too. The computing system 130 may then automatically alert the subject, via the interaction module, to start the XCS therapy at the preselected time, ensuring the therapy aligns with the subject's natural biological cycles.

Alternatively, the flowing rhythms are non-fixed rhythms that are more flexible and may be influenced by internal or external factors. The flowing rhythms may include ultradian rhythms, which may repeat multiple times within a 24-hour period such as the human sleep cycle that alternates between REM and non-REM stages. Additionally, heart rate variability (HRV) and respiratory patterns may also exhibit periodic fluctuations throughout the day. The regular and the flowing rhythms may also encompass a wider array of biological patterns, including additional cycles or patters that influence the subject's physiological processes, as detailed herein.

Once the rhythms are detected, the data may be transmitted to either a rhythmic synchronization module 710 or a scheduling module 715, depending on the nature or pattern of the rhythms. For flowing rhythms, which may be influenced by internal or external factors, the data may be sent to the rhythmic synchronization module 710. The rhythmic synchronization module 710 may adjust and synchronize the timing of the XCS to align with the dynamic biological rhythms of the subject, which may ensure that the XCS is delivered at the most appropriate moment for therapeutic effectiveness.

For regular rhythms, such as circadian rhythms or fixed cycles, the data may be transmitted to the scheduling module 715. The scheduling module 715 may allow the subject to preselect a specific time of a day for receiving the XCS based on the feedback from the feedback module 510. The computing system 130 may deliver the XCS at the preselected time on each day, in accordance with the subject's fixed biological rhythms. The scheduling module 715 may be designed to accommodate such fixed biological patterns, ensuring a predictable and consistent therapy schedule. Based on synchronization or scheduling process, the rhythmic parameters 720 may be generated and passed to the parameter selection module 120. In some aspects, the combination of both the rhythmic synchronization module 710 and the scheduling module 715 may collaboratively predict and refine the rhythmic parameters 720. By synchronizing and scheduling the XCS, the rhythmic dosing module 515 may help to reduce the risk of an overstimulation or an associated discomfort.

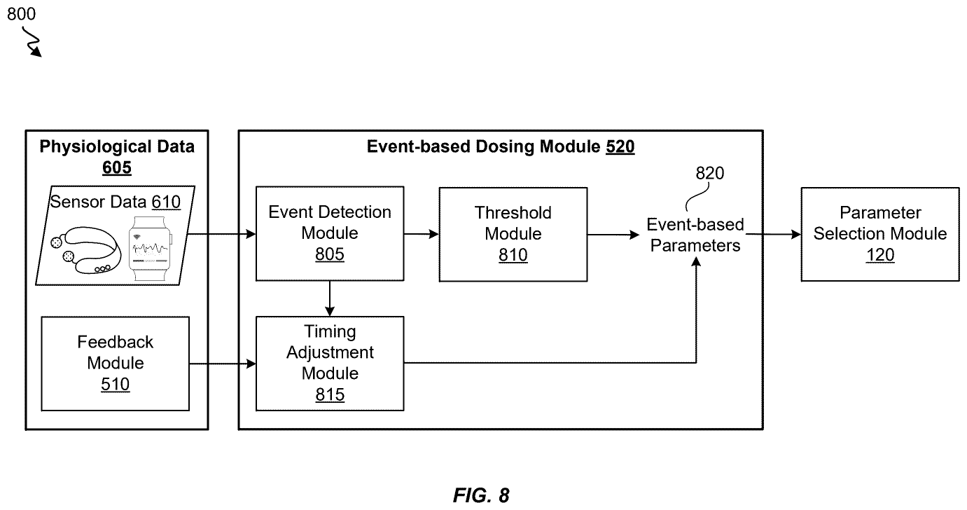

FIG. 8 shows an example illustration of an event-based dosing module 520 to configure a set of selection parameters based on the physiological data 605 of the subject in accordance with some aspects of the present disclosure. The event-based dosing module 520 may configure the event-based parameters 820 to deliver the XCS based on specific, identifiable events or triggers. The event-based dosing module 520 may prompt the system to deliver the XCS at the precise moment when the XCS therapy is most likely to be beneficial to the subject. For instance, the occurrence of specific physiological or behavioral events such as the subject exhibiting signs of stress, anxiety, cognitive decline, or other health indicators, may trigger the event-based dosing module 520. When the computing system 130 detects that the physiological data 605 indicates a potential relapse or increased stress levels (e.g., elevated heart rate, changes in brain activity or changes in vocal tone), the event-based dosing module 520 may immediately trigger the administration of the XCS.

Additionally, the event-based dosing module 520 may involve synchronizing the XCS with ongoing treatments such as pharmaceuticals, cognitive behavioral therapy, meditation and various neuromodulation therapies (e.g., deep brain stimulation, vagus nerve stimulation, TMS), as well as physical therapies, biofeedback, mindfulness practices, acupuncture, or other complementary treatments. By integrating such treatments with the XCS, the computing system 130 may ensure that the therapeutic benefits to the subject are amplified, either by activating the XCS at the optimal time during such treatments or by allowing the subject to trigger the XCS manually for enhanced effects.

An event detection module 805 of the event-based dosing module 520 may identify relevant events based on the physiological data 605 of the subject collected from one or more wearable assemblies. As disclosed herein, the events may be physiological triggers, or alternatively, the events may relate to the timing of other ongoing treatments or therapies. After an event may be detected by the event detection module 805, the data may be transmitted to a threshold module 810, if the event is a trigger. The threshold module 810 may evaluate whether the detected event meets a certain threshold limit, indicating a high-priority moment for an intervention. The threshold module 810 may check the intensity or significance of the event and decides if the XCS may be administered immediately, depending on the priority level assigned to the detected event. For example, if the physiological data 605 indicates a significant increase in stress or an impending cognitive decline, the threshold module 810 may trigger the delivery of the first XCS therapy.

Alternatively, if the detected event pertains to ongoing treatments (e.g., pharmaceuticals or cognitive therapy), the event data may be routed to a timing adjustment module 815. The timing adjustment module 815 may automatically adjust the timing of the XCS to better align it with the administration of other therapies based on the sensor data 610. In some aspects, the timing of the XCS may be manually controlled by the subject through the feedback from the feedback module 510. In such aspects, the subject may communicate the preferred time for the first XCS therapy, and the computing system 130 may adapt the XCS therapy schedule accordingly. The timing adjustment module 815 may ensure that the XCS may be delivered at an optimal moment, either in synchronization with ongoing treatments or based on the subject's input to enhance the therapeutic effect.

The threshold module 810 and the timing adjustment module 815 may compute the event-based parameters 820 that may then be transmitted to the parameter selection module 120 for further processing. The event-based dosing module 520 may help to provide a responsive and adaptive treatment regimen that considers the subject's immediate needs based on various biological and behavioral markers.

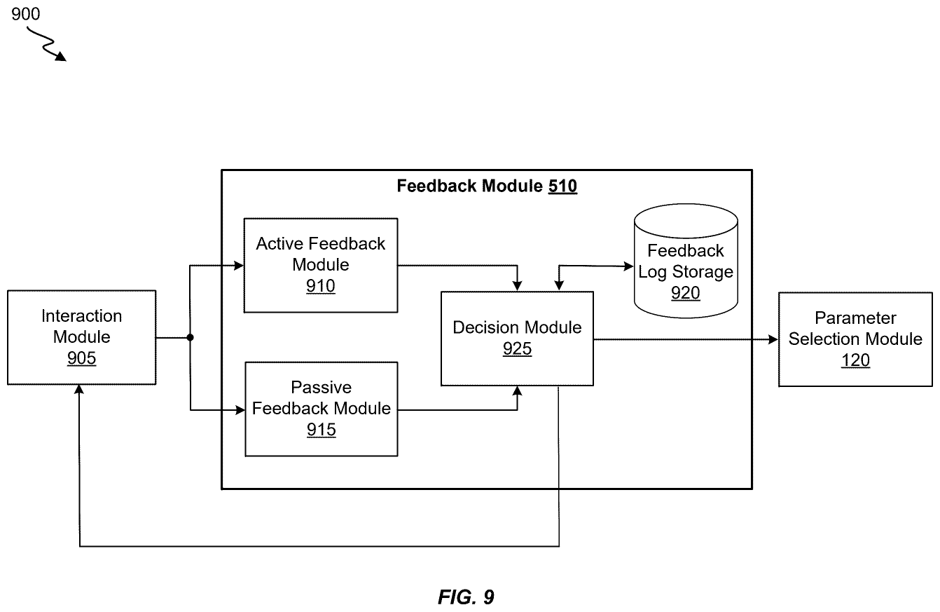

FIG. 9 shows an example illustration of the feedback module 510 as a part of the physiological data 605 of the subject and to revise the set of selection parameters to initiate the XCS therapy (e.g., initiating regenerated XCS) in accordance with some aspects of the present disclosure. The feedback module 510 may dynamically adjust the set of selection parameters based on real-time data about the physical and mental state of the subject. The feedback module 510 may also allow the subject to communicate a preferred time or schedule for receiving the XCS therapy through an interaction module 905.

The interaction module 905 may serve as a communication channel between the computing system 130 and the subject that may enable seamless and efficient data exchange. The interaction module 905 may allow for multiple modes of interaction, which may ensure that the computing system 130 may receive real-time data from the subject in various forms. For instance, the interaction module 905 may present questionnaires or prompts via the GUI to capture the subject's responses about the mental and physical state of the subject. The GUI may be presented on the computing system 130 or the wrist-worn device 200-B-one wearable of the one or more wearable devices. The interaction module 905 may also record voice logs, which may further be analyzed by the feedback module 510 to assess the emotional tone, word choice and other indicators related to the mental well-being of the subject. Additionally, the interaction module 905 may directly ask the subject about the schedule or time preferences. The interaction module 905 may also communicate to the subject when to wear the head-mounted device 205 to activate the stimulation electrode 210.

Once a response from the subject may be received, the interaction module 905 may process and organize the data that may then be appropriately forwarded to the relevant feedback mechanisms within the feedback module 510 for further analysis and actions. According to an example illustration, the data may be transmitted to an active feedback module 910 and/or a passive feedback module 915. The active feedback module 910 may process responses, forwarded by the interaction module 905, where the responses may include answers to questionnaires, prompts or direct queries about the mental and physical state of the subject. The responses may address issues such as mood, stress levels, cognitive function and/or other health indicators. Once the responses are received, the active feedback module 910 may employ mechanisms such as natural language processing (NLP), sentiment analysis or machine learning algorithms to analyze the content and context of the responses received. Such mechanisms may be utilized to predict the subject's mental state by detecting emotional tones, identifying key phrases and/or quantifying the severity of any signs of stress, anxiety or cognitive decline. After processing the responses, the data may be forwarded to a decision module 925.

In some aspects, the active feedback module 910 may generate and present questionnaires, prompts or assessments through the interaction module 905 to collect the responses from the subject. Such questionnaires or prompts may specifically be designed by the active feedback module 910 based on the subject's prior responses or ongoing therapeutic needs. In some aspects, a feedback log storage 920 may store the questionnaires, prompts or assessments that may be presented to the subject.

The passive feedback module 915 may access and monitor the mental and emotional state of the subject through a passive data collection. The interaction module 905 may record the voice of the subject at all times via the one more wearable devices (e.g., a microphone integrated in the wrist-worn device 200-B or the head-mounted device 205). Such continuous voice recordings may be logged and stored for analysis, which provides real-time data that may be processed without requiring an active participation of the subject. The recorded voice logs may be stored in the feedback log storage 920 within the feedback module 510 or within the passive feedback module 915. The feedback log storage 920 may serve as the repository for all data related to the feedback module 510 including voice logs, questionnaire responses, subject preferences and/or decisions made based on the processed data. Such comprehensive storage may allow efficient tracking and analysis of all relevant feedback information, which may ensure that the computing system 130 or the feedback module 510 can make personalized, informed decisions about delivering the XCS therapy.

The passive feedback module 915 may process the voice data by examining various features including intonation, word choice, speech patterns and pitch variation (e.g., a rise and fall in the pitch of the voice). Such features may be analyzed using technologies such as speech recognition, NLP and/or emotion detection algorithms. Within such technologies, the speech recognition algorithms may be employed to transcribe and interpret the spoken language, which may enable the passive feedback module 915 to identify key phrases and words that may indicate specific emotional states or cognitive conditions. For instance, a rapid or erratic speech pattern may suggest stress or anxiety, while slower or more fragmented speech might indicate a cognitive decline. Additionally, the NLP techniques may be used to assess the contextual meaning of the subject's words, which may reveal underlying emotional distress, feelings of sadness or signs of a positive mental state. To gain a deeper understanding of the mental state of the subject, the emotion detection algorithms may be used to evaluate subtle variations in the subject's voice including intonation, speech tempo and pitch variation, all of which may indicate emotional states such as stress, anxiety, happiness or frustration.

Based on the analysis, the passive feedback module 915 may determine the current emotional or mental condition of the subject. If signs of distress or mental health deterioration are detected, the passive feedback module 915 may pass on recommendations to trigger the XCS therapy, which may involve adjusting the set of selection parameters, modifying the waveform parameters 620 or scheduling the second XCS therapy to provide therapeutic intervention at a most appropriate time. The passive feedback module 915 may transmit the recommendations to the decision module 925.

The decision module 925, based on data received from the active feedback module 910, the passive feedback module 915 and the feedback log storage 920, may determine the appropriate course of action for the subject's therapy. By analyzing the mental state of the subject, the decision module 925 may decide whether the subject may wear the head-mounted device 205 to receive the second XCS therapy or if a further intervention may be necessary. The decision module 925 may communicate to the subject, via the interaction module 905, to wear the head-mounted device 205 for the XCS therapy and to provide the selected time for the therapy based on the physiological data of the subject. It may also notify the subject if the second XCS therapy is required and confirm if the preferred schedule is approved, indicating any adjustments made by the clinician or medical provider. Subsequently it may also instruct the subject to fill out a questionnaire if needed and/or alert the subject to physiological triggers or alerts detected from the one or more wearable assemblies.

The decision module 925 may decide the further course of action based on one or more conditions: (1) recovery or remission, which may lead to a reduction or stabilization in the set of selection parameters to avoid an over-treatment; (2) entering relapse, which may prompt an increase in the set of selection parameters to provide an intensive therapeutic intervention; and (3) a steady state, which may necessitate not to modify the set of selection parameters and may allow for a consistent therapy without unnecessary adjustments.

The one or more conditions refer to distinct stages in the subject's mental and physical well-being, each influencing the therapeutic approach. The recovery or remission may occur when the symptoms or distress of the subject may significantly decrease, and/or the mental or emotional state may show improvement or stability. During the recovery, the set of selection parameters may be adjusted to decrease or stabilize the intensity of the XCS therapy to prevent an over-treatment and allow the subject to maintain this improved state. In contrast, the relapse may refer to a phase where the subject exhibits signs of mental or emotional decline (e.g., increased stress, anxiety or cognitive difficulties). During relapse, the set of selection parameters may be increased to intensify the XCS therapy, aiming to counteract the decline and provide an enhanced support to the subject. The steady state condition may suggest that the subject's mental and physical state is stable indicating that is neither improving nor deteriorating. In such a state, the set of selection parameters may be kept unchanged, as there is no immediate need for adjustments, which may allow consistent, ongoing therapy to maintain the current condition of the subject.

In some aspects, the data from the decision module 925 may directly be used by the XCS-EEG module 505 to adjust the waveform parameters 620 associated with each stimulation of the XCS therapy. In some other aspects, according to the example illustration 900, the data from the decision module 925 may modify the set of selection parameters within the parameter selection module 120, which may then be transmitted to the XCS-EEG module 505 to adjust the waveform parameters 620, if necessary.

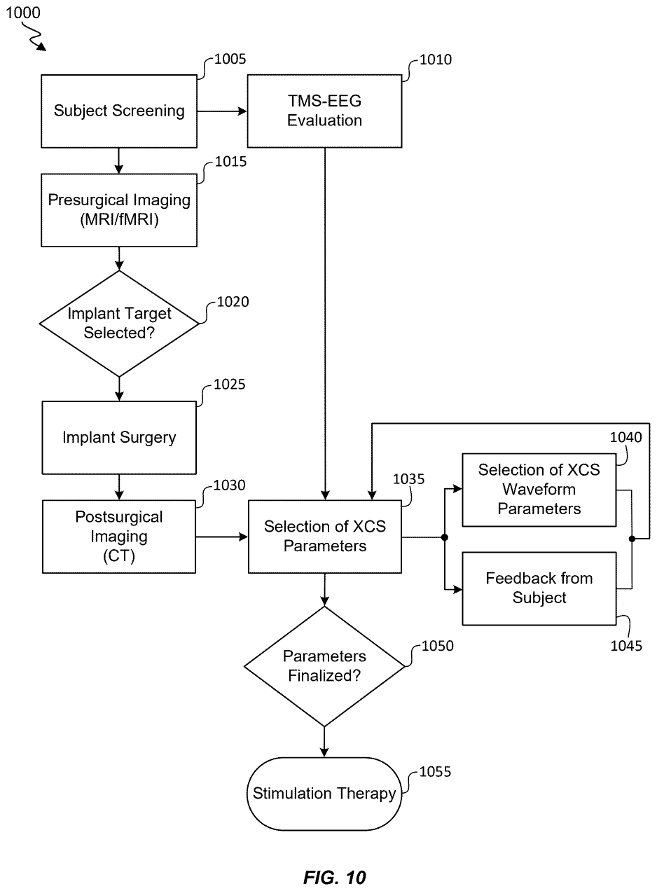

FIG. 10 illustrates an exemplary workflow 1000 to initiate the XCS therapy to the subject based on the set of selection parameters in accordance with some aspects of the present disclosure. At block 1005, the screening module 105 may screen the subject by evaluating the physiological conditions, based on the physiological data 605 of the subject. The physiological data 605 may be collected through the one or more wearable assemblies (e.g., the head-mounted device 205 or the wrist-worm device 200-B), cognitive assessments, neuroimaging, diagnostic tests etc. The screening of the subject may analyze whether the subject exhibits a potential disorder or is at a risk of developing one.

At block 1010, a TMS-EEG evaluation may be performed by the TMS-EEG evaluation module 115. The TMS-EEG evaluation module 115 may deliver the TMS to the target region of the subject and record the evoked responses to configure the initial parameters (e.g., initial stimulation amplitude) associated with the stimulation electrode 210. The initial stimulation amplitude of the set of selection parameters may then be used to initiate the XCS.

Based on the physiological condition of the subject, the stimulation electrode 210 may be implanted on or inside the subject. At block 1015, the presurgical imaging may be performed by the XCS implantation module 110 to select the target region for the implantation of the stimulation electrode 210. The presurgical imaging may include, but is not limited to, MRI, fMRI, CT scans or other relevant imaging techniques that may help identify and precisely locate the cortical region. In some aspects, MRI, fMRI may be collected by the screening module 105 as a part of the physiological data 605 and passed down to the computing system 130 for further processing.

At block 1020, the computing system 130 may decide a cortical region for implanting the stimulation electrode in the subject based on the collected physiological data 605 and presurgical imaging. Once the cortical region is selected, at block 1025, the stimulation electrode may be implanted into the skull of the subject through a burr hole procedure.

At block 1030, postsurgical imaging may be performed to verify the proper placement of the stimulation electrode 210. The postsurgical imaging may ensure that the placement of the stimulation electrode 210 may meet therapeutic requirements before proceeding with the XCS therapy.

At block 1035, initial stimulation amplitude associated with the set of selection parameters may be received from the block 1010 (e.g., the TMS-EEG evaluation module 115), and the stimulation electrode 210. The set of selection parameters may comprise waveform parameters 620 configured by XCS-EEG module 505. In some aspects, the set of selection parameters may be configured by rhythmic parameters 720 that may be determined by the rhythmic dosing module 515. In some aspects, the set of selection parameters may be configured by event-based parameters 820 that may be determined by the event-based dosing module 520. The set of selection parameters may be readjusted based on the feedback(e.g., active or passive feedback data) from the subject, at block 1045. At block 1040, the waveform parameters 620 of the XCS may be modified based on the feedback from the feedback module 510 (e.g., from block 1045). Such modified waveform parameters may then be fed back to the block 1035, where the parameter selection module 120 may configure the set of selection parameters to regenerate the XCS.

At block 1050, the computing system 130 may reconfigure to determine whether the set of selection parameters is finalized, which may be performed by the parameter selection module 120. Based on the decision, at block 1055, the set of selection parameters may be forwarded to the stimulation generator 125 to regenerate the XCS. The regenerated XCS may be initiated, via the stimulation electrode 210, to the cortical region of the subject.

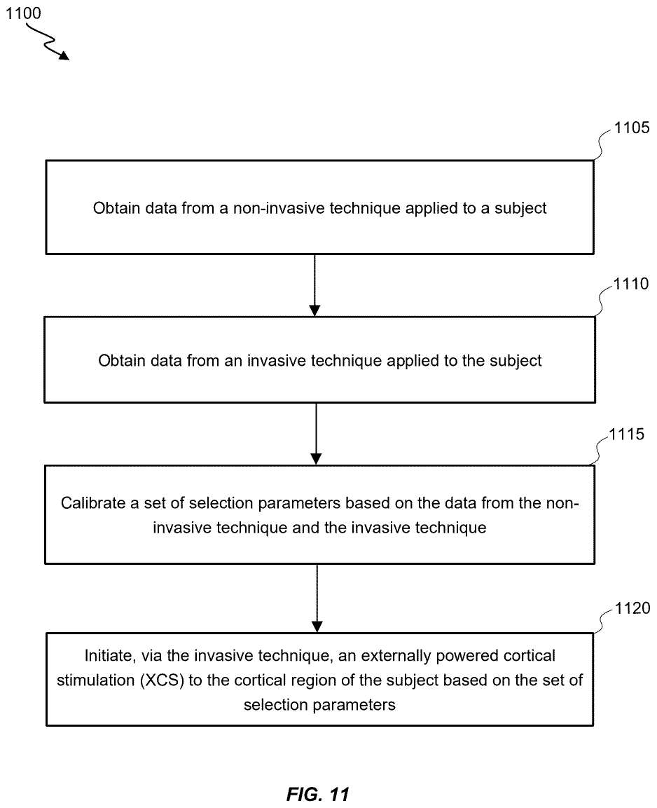

FIG. 11 illustrates an exemplary workflow 1100 to calibrate the set of selection parameters associated with the invasive technique (e.g., the XCS) based on the non-invasive technique (e.g., the TMS) in accordance with some aspects of the present disclosure. The blocks in the exemplary workflow 1100 are illustrated in a specific order, while the order may be modified, for example, some blocks may be performed before others, and some blocks may be performed simultaneously. The block may be performed by hardware, software, or a combination thereof.

At block 1105, the data may be obtained from the non-invasive technique comprising determining motor threshold 405 based on a motor response, determining recruitment curves associated with the TMS and identifying normalized peaks of the recruitment curves associate with the TMS at the motor threshold 405. The motor threshold may be determined by applying a stimulus delivered by the TMS coils 305 to a motor cortex region of the subject and identifying the intensity of the stimulus that evokes a motor response in a controlled body part (e.g., thumb, hand) with 50% probability. Relocating the TMS coils 305 over a cortical region, other than the motor cortex, (for example) left DLPFC to map recruitment curves by varying stimulation amplitudes between 0% and 100% and recording the resulting evoked responses using the EEG electrodes (e.g., sensor electrodes 215a-b). From the evoked responses, normalized peaks (e.g., TMS-induced normalized peaks 315a-e) at the motor threshold 405 may be identified for the TMS.

At block 1110, the data may be obtained from the invasive technique comprising implanting the stimulation electrode 210, determining recruitment curves associated with the XCS and identifying normalized peaks of the recruitment curves associate with the XCS at the motor threshold 405. Recruitment curves may be mapped by sweeping the stimulation amplitudes between 0 and 15 mA, and evoked responses may be recorded using the EEG electrodes (e.g., the sensor electrodes 215a-b), From the recruitment curves, normalized peaks (e.g., XCS-induced normalized peaks 330a-e) associated with the XCS may be identified at the motor threshold 405.

At block 1115, after obtaining data from the non-invasive technique and the invasive technique, a set of selection parameters associated with the invasive technique may be calibrated. For the calibration, the normalized peaks of the recruitment curves of the TMS and the XCS may be matched to identify the stimulation amplitudes at which the evoked response peaks from the non-invasive technique align most closely with those generated by the invasive technique. Based on matching of the normalized peaks, an initial stimulation amplitude for the invasive technique may be determined. The initial stimulation amplitude may be associated with the set of selection parameters.

At block 1120, an XCS may be generated by the stimulator generator 125 based on the set of selection parameters. After generation, the XCS may be delivered via the stimulation electrode 210 to the cortical region of the subject.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability or configuration of the disclosure. Rather, the present description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the present description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1. A computer-implemented method including:
obtaining data from a non-invasive technique applied to a subject including:
determining a motor threshold based on a motor response of the subject by delivering a stimulus using the non-invasive technique over a motor cortex region;
determining recruitment curves associated with the non-invasive technique by sweeping stimulation amplitudes of the non-invasive technique over a cortical region and recording evoked responses via electroencephalography (EEG) electrodes integrated in a wearable assembly of one or more wearable assemblies; and
identifying normalized peaks of the recruitment curves associated with the non-invasive technique at the motor threshold;
obtaining data from an invasive technique applied to the subject including:
determining recruitment curves associated with the invasive technique by sweeping stimulation amplitudes of the invasive technique over the cortical region and recording evoked responses by the EEG electrodes integrated in the wearable assembly of the one or more wearable assemblies; and
identifying normalized peaks of the recruitment curves associated with the invasive technique at the motor threshold;
calibrating a set of selection parameters based on the data from the non-invasive technique and the invasive technique, comprising:
matching the normalized peaks of the recruitment curves associated with the non-invasive technique and the invasive technique; and
configuring, based on the matched normalized peaks, an initial stimulation amplitude for the invasive technique associated with the set of selection parameters; and
initiating, via the invasive technique, an externally powered cortical stimulation (XCS) to the cortical region of the subject based on the set of selection parameters.

2. The computer-implemented method of claim 1, wherein the non-invasive technique comprises a transcranial magnetic stimulation (TMS) delivered using TMS coils.

3. The computer-implemented method of claim 1, wherein the invasive technique comprises implanting a stimulation electrode over the cortical region of the subject and using the stimulation electrode to deliver the XCS.

4. The computer-implemented method of claim 1, wherein the one or more wearable assemblies further facilitates:
collecting physiological data of the subject that comprises one or more of: neural data, cardiovascular data, respiratory data, movement and activity data, environmental data, demographic data, anthropometric data, comorbidities data, medical imaging data, lifestyle indicators data, feedback or a combination thereof; and
externally powering, via a wearable assembly of the one or more wearable assemblies, the stimulation electrode associated with the invasive technique, wherein the wearable assembly of the one or more wearable assemblies is a head-mounted device having the EEG electrodes.

5. The computer-implemented method of claim 1, wherein initiating the XCS to the cortical region of the subject based on the set of selection parameters is further configured using rhythmic parameters, wherein the rhythmic parameters are derived from one or more biological rhythms associated with physiological data of the subject.

6. The computer-implemented method of claim 1, wherein initiating the XCS to the cortical region of the subject based on the set of selection parameters is further configured using event-based parameters, wherein the event-based parameters are derived from one or more events associated with the physiological data of the subject to trigger the XCS either independently or in conjunction with other therapies.

7. The computer-implemented method of claim 1, further including:
adjusting, based on the feedback derived from the physiological data of the subject, the set of selection parameters, wherein the set of selection parameters are configured to predict, based on the feedback, whether the subject is recovering, entering relapse or is in a stable state;
regenerating an XCS based on the adjusted set of selection parameter; and
initiating, via the invasive technique, the regenerated XCS to the cortical region of the subject.

8. The computer-implemented method of claim 7, wherein the feedback derived from the physiological data of the subject further includes an active feedback and a passive feedback.

9. The computer-implemented method of claim 1, wherein the cortical regions comprise the motor cortex region, sensory cortex, visual cortex, auditory cortex, prefrontal cortex including left and right dorsolateral prefrontal cortex (DLPFC) or language areas including Broca's and Wernicke's areas.

10. A system comprising:
one or more processors;
one or more non-transitory computer-readable media storing instructions, which, when executed by the system, cause the system to perform part a set of actions including:
obtaining data from a non-invasive technique applied to a subject including:
determining a motor threshold based on a motor response of the subject by delivering a stimulus using the non-invasive technique over a motor cortex region;
determining recruitment curves associated with the non-invasive technique by sweeping stimulation amplitudes of the non-invasive technique over a cortical region and recording evoked responses via electroencephalography (EEG) electrodes integrated in a wearable assembly of one or more wearable assemblies; and
identifying normalized peaks of the recruitment curves associated with the non-invasive technique at the motor threshold;
obtaining data from an invasive technique applied to the subject including:
determining recruitment curves associated with the invasive technique by sweeping stimulation amplitudes of the invasive technique over the cortical region and recording evoked responses by the EEG electrodes integrated in the wearable assembly of the one or more wearable assemblies; and
identifying normalized peaks of the recruitment curves associated with the invasive technique at the motor threshold;
calibrating a set of selection parameters based on the data from the non-invasive technique and the invasive technique, comprising:
matching the normalized peaks of the recruitment curves associated with the non-invasive technique and the invasive technique; and
configuring, based on the matched normalized peaks, an initial stimulation amplitude for the invasive technique associated with the set of selection parameters; and
initiating, via the invasive technique, an externally powered cortical stimulation (XCS) to the cortical region of the subject based on the set of selection parameters.

11. The system of claim 10, wherein the non-invasive technique comprises a transcranial magnetic stimulation (TMS) delivered using TMS coils.

12. The system of claim 10, wherein the invasive technique comprises implanting a stimulation electrode over the cortical region of the subject and using the stimulation electrode to deliver the XCS.

13. The system of claim 10, wherein the one or more wearable assemblies further facilitates:
collecting physiological data of the subject that comprises one or more of: neural data, cardiovascular data, respiratory data, movement and activity data, environmental data, demographic data, anthropometric data, comorbidities data, medical imaging data, lifestyle indicators data, feedback or a combination thereof; and
externally powering, via a wearable assembly of the one or more wearable assemblies, the stimulation electrode associated with the invasive technique, wherein the wearable assembly of the one or more wearable assemblies is a head-mounted device having the EEG electrodes.

14. The system of claim 10, wherein initiating the XCS to the cortical region of the subject based on the set of selection parameters is further configured using rhythmic parameters, wherein the rhythmic parameters are derived from one or more biological rhythms associated with physiological data of the subject.

15. The system of claim 10, wherein initiating the XCS to the cortical region of the subject based on the set of selection parameters is further configured using event-based parameters, wherein the event-based parameters are derived from one or more events associated with the physiological data of the subject to trigger the XCS either independently or in conjunction with other therapies.

16. A computer-program product tangibly embodied in a non-transitory machine-readable storage medium, including instructions configured to cause one or more data processors to perform a set of actions including:
obtaining data from a non-invasive technique applied to a subject including:
determining a motor threshold based on a motor response of the subject by delivering a stimulus using the non-invasive technique over a motor cortex region;
determining recruitment curves associated with the non-invasive technique by sweeping stimulation amplitudes of the non-invasive technique over a cortical region and recording evoked responses via electroencephalography (EEG) electrodes integrated in a wearable assembly of one or more wearable assemblies; and
identifying normalized peaks of the recruitment curves associated with the non-invasive technique at the motor threshold;
obtaining data from an invasive technique applied to the subject including:
determining recruitment curves associated with the invasive technique by sweeping stimulation amplitudes of the invasive technique over the cortical region and recording evoked responses by the EEG electrodes integrated in the wearable assembly of the one or more wearable assemblies; and
identifying normalized peaks of the recruitment curves associated with the invasive technique at the motor threshold;
calibrating a set of selection parameters based on the data from the non-invasive technique and the invasive technique, comprising:
matching the normalized peaks of the recruitment curves associated with the non-invasive technique and the invasive technique; and
configuring, based on the matched normalized peaks, an initial stimulation amplitude for the invasive technique associated with the set of selection parameters; and
initiating, via the invasive technique, an externally powered cortical stimulation (XCS) to the cortical region of the subject based on the set of selection parameters.

17. The computer-program product of claim 16, wherein the non-invasive technique comprises a transcranial magnetic stimulation (TMS) delivered using TMS coils.

18. The computer-program product of claim 16, wherein the invasive technique comprises implanting a stimulation electrode over the cortical region of the subject and using the stimulation electrode to deliver the XCS.

19. The computer-program product of claim 16, wherein the one or more wearable assemblies further facilitates:
collecting physiological data of the subject that comprises one or more of: neural data, cardiovascular data, respiratory data, movement and activity data, environmental data, demographic data, anthropometric data, comorbidities data, medical imaging data, lifestyle indicators data, feedback or a combination thereof; and
externally powering, via a wearable assembly of the one or more wearable assemblies, the stimulation electrode associated with the invasive technique, wherein the wearable assembly of the one or more wearable assemblies is a head-mounted device having the EEG electrodes.

20. The computer-program product of claim 16, wherein initiating the XCS to the cortical region of the subject based on the set of selection parameters is further configured using rhythmic parameters, wherein the rhythmic parameters are derived from one or more biological rhythms associated with physiological data of the subject.
